(19) United States
(12) Patent Application Publication
(10) Pub. No.: US 2004/0220593 A1
(43) Pub. Date: Nov. 4, 2004

(54) Restraining clip for mitral valve repair

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950

(73) Assignee: Secant Medical, LLC, Perkasie, PA

(21) Appl. No.: 10/827,552

(22) Filed: Apr. 19, 2004

Related U.S. Application Data
(60) Provisional application No. 60/467,215, filed on May 1, 2003.

(51) Int. Cl. A61F 2/24

(52) U.S. Cl. 606/151; 623/2.1

(57) ABSTRACT

A clip for repair of a heart valve is disclosed. The clip has a plurality of legs joined at one end to a common attachment, the opposite ends of the legs being free to move. The legs are resilient and flexible, allowing the free ends to be separated to receive the leaflets of a valve between them. The legs are resiliently biased so as to close upon the leaflets. Hooks positioned at the free ends engage the leaflets and attach the legs to them. Thus positioned, the clip restrains the motion of the leaflets relatively to one another to prevent valve prolapse. Legs formed of loops or having spiral shapes are feasible. The common attachment may comprise a threaded ferrule, a bent or looped wire segment as well as a cylindrical shell or a ring.

### FIELD OF THE INVENTION

The invention concerns a device and a method for effecting the repair of a bicuspid heart valve in a manner having the effect of an Alfieri leaflet stitch.

### BACKGROUND OF THE INVENTION

As shown in FIG. 1, the left atrioventricular or mitral valve 1 is a bicuspid (two-leaflet) valve positioned in the orifice 2 between the left atrium 3 and left ventricle 4 of the heart 5. Oxygen rich blood 6 flows from the lungs via pulmonary veins 7 to the left atrium 3, past the mitral valve 1 and into the left ventricle 4 where it is pumped to the aorta 8 for further distribution to the body. The mitral valve acts as a one-way valve, the leaflets 9 and 11 closing against one another (also known as coaptation) in response to the pressure increase in the left ventricle 4 caused when it contracts to pump the blood through the aorta 8. A healthy mitral valve prevents a back flow of blood through the left atrium 3 to the lungs.

Of the various valves in the heart, the mitral valve is most vulnerable to disease and suffers from atrioventricular valvular incompetence whereby the mitral valve seals incompletely. This allows blood to regurgitate into the left atrium upon contraction of the left ventricle. The diseased leaflets of the valve undergo scarring and shortening which are one cause the valvular incompetence. Other causes include abnormal elongation of the chordae tendineae (tendinous cords attached to the free edges of the leaflets to prevent prolapse), as well as rigidity, deformity and retraction of the leaflets associated with rheumatic heart disease.

Due to the low success rate associated with mitral valve replacement, atrioventricular valvular incompetence is preferably treated by mitral valve repair requiring open heart surgery. This treatment is extremely invasive, requires that the heart be stopped and the patient put on cardiopulmonary bypass and often leads to post-operative complications.

Mitral valve repair is effected by joining the valve leaflets or otherwise restraining their motion relative to one another at a point along their free edges. In a healthy valve, these edges normally seal against each other (coapt) to prevent back flow of blood. However, in a diseased mitral valve, the free edges may prolapse into the left atrium and allow a back flow of blood. The joining or restraint is effectively provided by the use of an Alfieri leaflet stitch joining the leaflets at a point along their free edges, thereby constraining the motion of the leaflets relatively to one another at that point. Such a constraint allows the leaflets to open on either side of the Alfieri stitch to admit blood to the left ventricle but prevents prolapse and allows effective sealing of the valve.

Clearly, there is a need for effecting the restraint of the valve leaflets of the mitral valve in the manner of an Alfieri leaflet stitch for the treatment of mitral valve disorders which is less invasive than open heart surgery.

### SUMMARY OF THE INVENTION

The invention concerns a clip for repair of the leaflets of a heart valve by joining the leaflets together or restraining the motion of the leaflets relatively to one another substantially at a point along the free edges of the leaflets. The clip comprises a plurality of resilient, flexible legs arranged substantially lengthwise adjacent to one another. Each of the legs has a first end adapted to attach to one of the leaflets and a second end positioned opposite the first end. A common attachment is located at the second ends of the legs. The second ends are connected, attached together or otherwise fixed relatively to one another at the common attachment. The first ends of the legs are flexibly movable away from one another for receiving the leaflets therebetween. The legs, furthermore, are resiliently biased toward each other for engagement of each of the first ends with one of the leaflets for attaching the clip to the valve.

The invention also concerns a method of repairing the leaflets of a heart valve by restraining the motion of the leaflets substantially at a point along their free edges. The method comprises the steps of:

(a) providing a clip comprising a plurality of resilient, flexible legs arranged substantially lengthwise adjacent to one another and resiliently biased toward one another, each of the legs having a first end adapted to attach to one of the leaflets and a second end, positioned opposite the first end, the clip further comprising a common attachment located at the second ends of the legs, the second ends being fixed relatively to one another at the common attachment;

(b) flexibly moving the first ends of the legs away from one another;

(c) positioning the legs with the leaflets therebetween and the common attachment adjacent to the free edge of the leaflets;

(d) allowing each of the first ends to be biased into engagement with one of the leaflets and thereby attaching the clip to the valve, the common attachment being positioned substantially adjacent to the free edge of the leaflets and defining the point at which the motion of the leaflets is substantially restrained.

### BRIEF DESCRIPTION OF THE DRAWINGS

FIG. 1 is a partial sectional view of a human heart;

FIG. 2 is a detailed view of a portion of the heart showing the invention on an enlarged scale;

FIG. 2A is a perspective view of a clip according to the invention;

FIG. 2B is a partial sectional view of the clip shown in FIG. 2;

FIG. 3A is a perspective view of an alternate embodiment of the clip according to the invention;

FIG. 3B is a partial sectional view of the claim shown in FIG. 3A;

FIGS. 4-15B are various embodiments of clips according to the invention; and

FIGS. 16-20 illustrate the method of effecting mitral valve repair using a clip according to the invention.

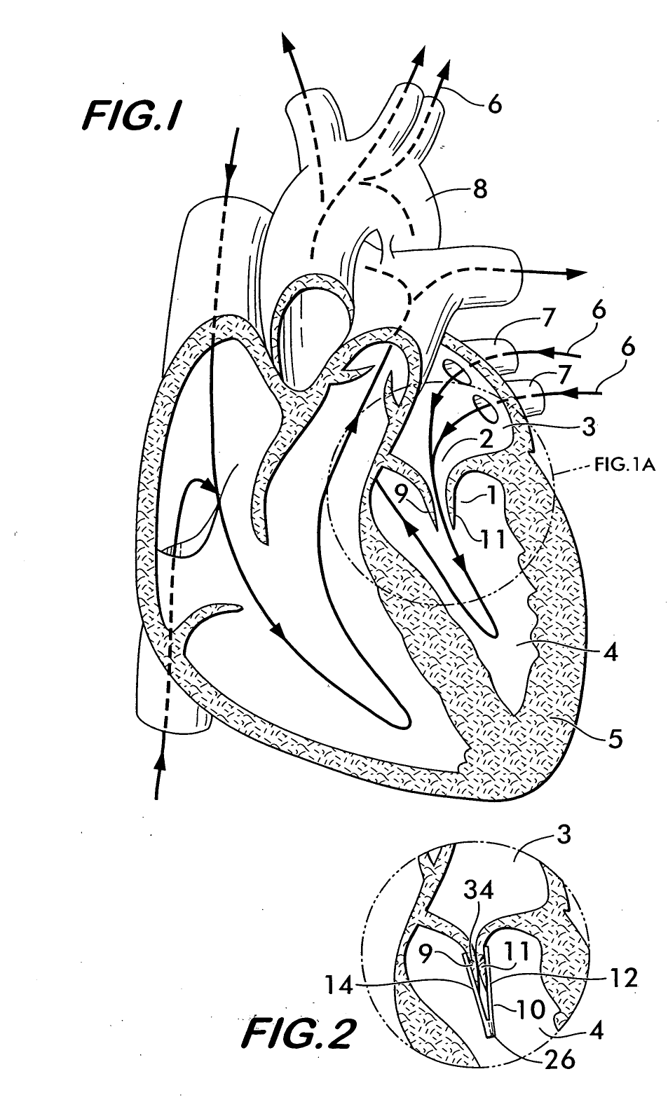

### DETAILED DESCRIPTION OF THE PRESENTLY PREFERRED EMBODIMENTS

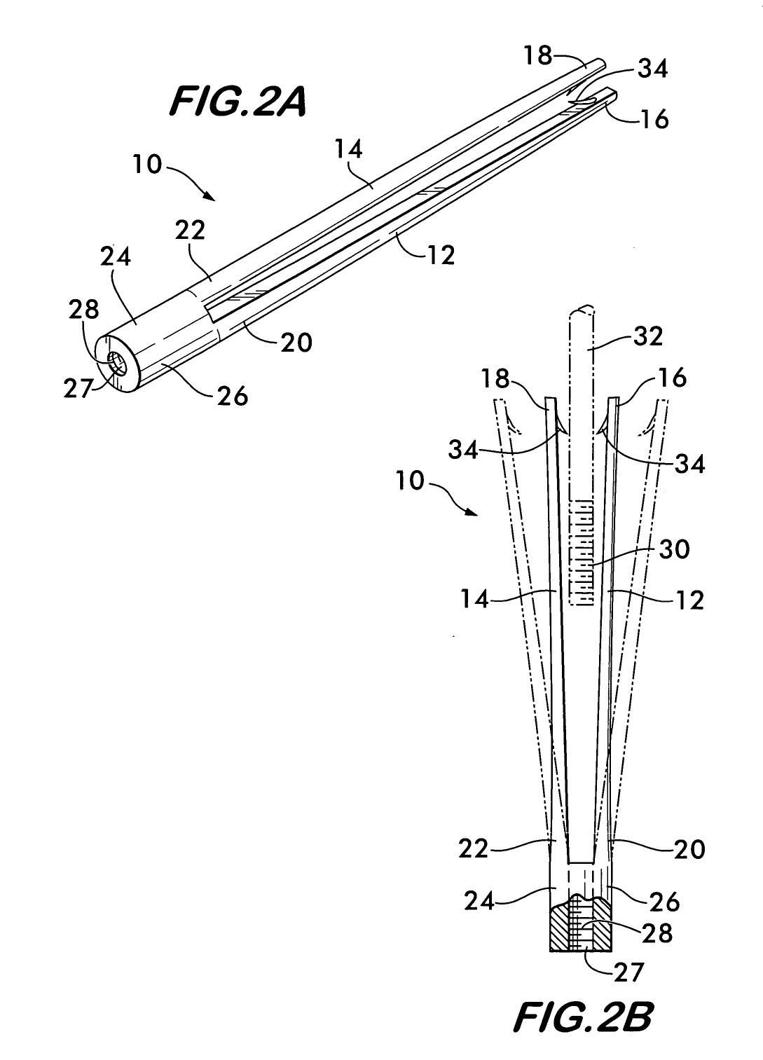

FIG. 2A shows an embodiment of a clip 10 for mitral valve repair according to the invention. Clip 10 has a pair of flexible, resilient legs 12 and 14 arranged lengthwise adjacent to one another. The legs 12 and 14 each have respective free ends 16 and 18 and respective root ends 20 and 22 arranged opposite to the free ends. The root ends are connected to a common attachment 24 which restrains the motion of the root ends 20 and 22 relatively to one another. The common attachment 24 may take a variety of forms, some of which are described below, but in FIGS. 2A and 2B, the common attachment comprises an elongated ferrule 26 having a lengthwise extending bore 27 with screw threads 28 on its inside surface for receiving a threaded tip 30 of an insertion device 32. Clip 10 is temporarily mountable on tip 30 and passed through a catheter lumen to insert the clip on the valve leaflets as described in detail below.

The free ends 16 and 18 are adapted to attach to the valve leaflets of the mitral valve of the heart. As shown in FIGS. 2A and 2B, the attachment means comprises a sharp hook 34 on each leg 12 and 14, the hooks extending angularly from the legs in a reverse direction lengthwise of the legs. As shown in FIG. 2, the hooks 34 act as barbs which engage the tissue of the mitral valve leaflets 9 and 11 to secure the clip 10 in place. Legs 12 and 14 are flexible, allowing the free ends 16 and 18 to be moved apart as shown in broken line in FIG. 2B to receive the leaflets of the mitral valve between them, the legs also being resiliently biased to force the hooks 34 into engagement with the valve tissue when the clip is positioned within the heart with the mitral valve leaflets between the free ends of the legs as shown in FIG. 2 and further described below.

Preferably, clips such as clip 10 are made of bio-compatible material having significant flexibility and high yield stress to provide the resilient, flexible qualities advantageous for the legs. Metals such as nitinol, elgiloy and titanium are preferred, although plastic materials such as polystyrene, polypropylene and polytetrafluoroethylene are also feasible. A practical clip length is approximately 1/6 the length of the valve leaflet as measured from the free edge to the root of the leaflet, although other clip lengths are also feasible.

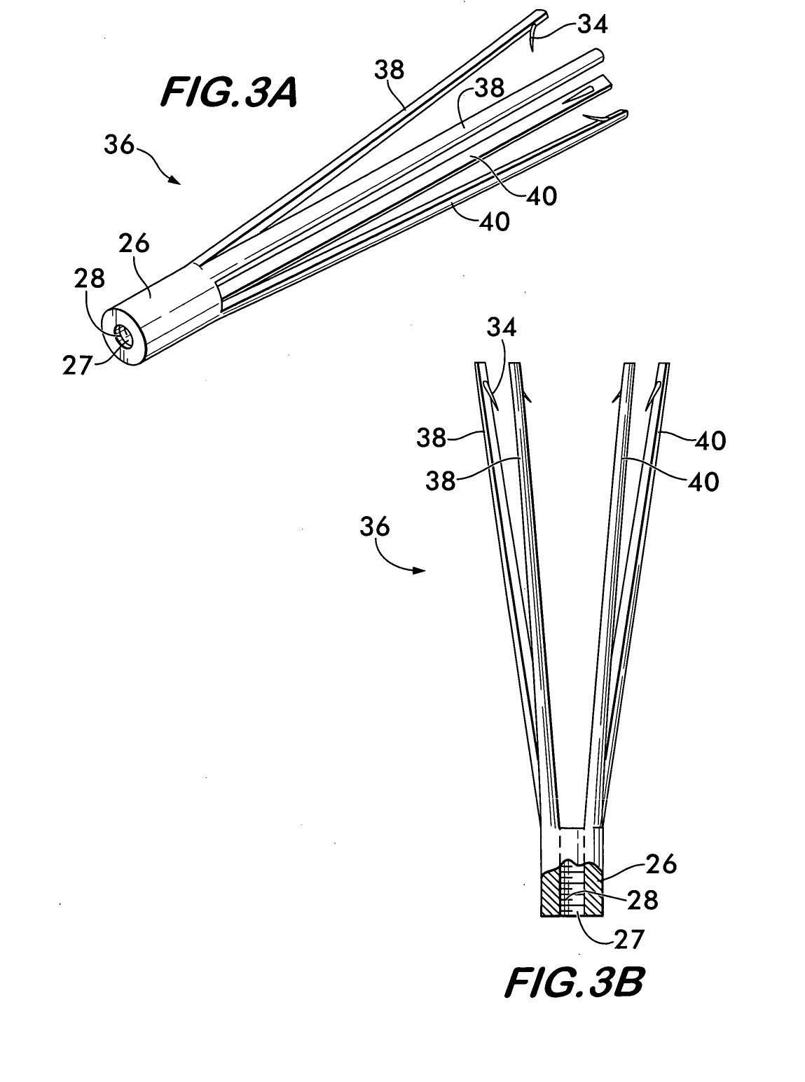

FIGS. 3A and 3B show another embodiment of a clip 36 according to the invention, the clip again retaining the threaded ferrule 26 as the common attachment but having leg pairs 38 and 40 arranged lengthwise adjacent to each other and attachable to each leaflet, leg pair 38 being secured to one leaflet and leg pair 40 being secured to the other when installed. Using multiple legs per leaflet, such as in pairs, triples and so forth, provides more attachment points for securing the clip to the valve, thereby distributing the load and lowering the force borne by any one leg or hook to provide a more reliable attachment of the clip to the valve.

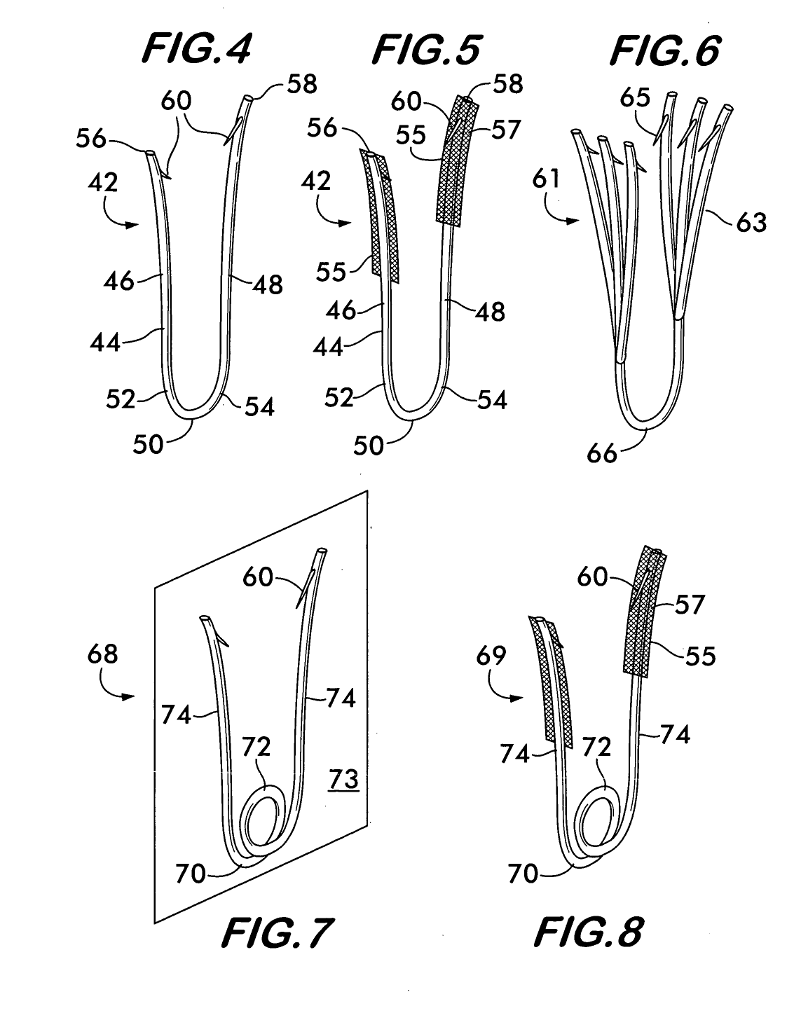

Another clip embodiment 42 is illustrated in FIG. 4. Clip 42 is comprised of drawn wire 44, the legs 46 and 48 being attached to a common attachment formed by a reverse bend segment of the wire 50, the bend segment 50 preferably being integrally formed with root ends 52 and 54 of the legs. The free ends 56 and 58 of the legs have hooks 60 for engagement with the tissue of the valve leaflets. As shown in FIG. 5, substrates 55 may be added lengthwise along each leg 46 and 48. The substrates 55 preferably comprise interlaced filamentary members of a bio-compatible material such as polyester, polypropylene and nylon and are adhesively bonded to the legs. The filamentary members may be interlaced by weaving, braiding or knitting and provide a lattice having pores 57 that promote the ingrowth of tissue from the mitral valve leaflets thereby providing further attachment of the clip 42 to the leaflets. Naturally porous materials, such as expanded polytetrafluoroethylene may also be used to form the substrates 55. Tissue ingrowth may be further encouraged by coating the substrates 55 with compounds such as thrombin and collagen which elicit a healing or clotting function from living tissue.

FIG. 6 shows a clip embodiment 61 comprising multiple legs 63 formed of wire stock, the legs having respective hooks 65 and being connected together at a common attachment 66 formed from a wire segment. The multiple legs 63 serve to distribute the forces required to keep the clip attached to the valve leaflets during valve opening and closing and may be integrally formed with, as well as welded, swaged, soldered, brazed or adhesively bonded to the common attachment 66.

FIG. 7 shows a clip embodiment 68 formed of wire and having a common attachment 70 comprising one or more continuous loops 72. The loops provide control over the biasing of the legs 74, as well as their flexibility. Preferably, loops 72 are oriented substantially parallel to the plane 73 defined by the legs 74, the legs being integrally formed with the loop or loops as a continuous wire segment. Loops 72 also provide stress relief to the common attachment by providing a greater length of wire over which the bending stresses may be distributed, thus, increasing the fatigue life of the clip 68. Loops 72 may also be combined on a clip with porous substrates 55 as illustrated in the embodiment 69 shown in FIG. 8.

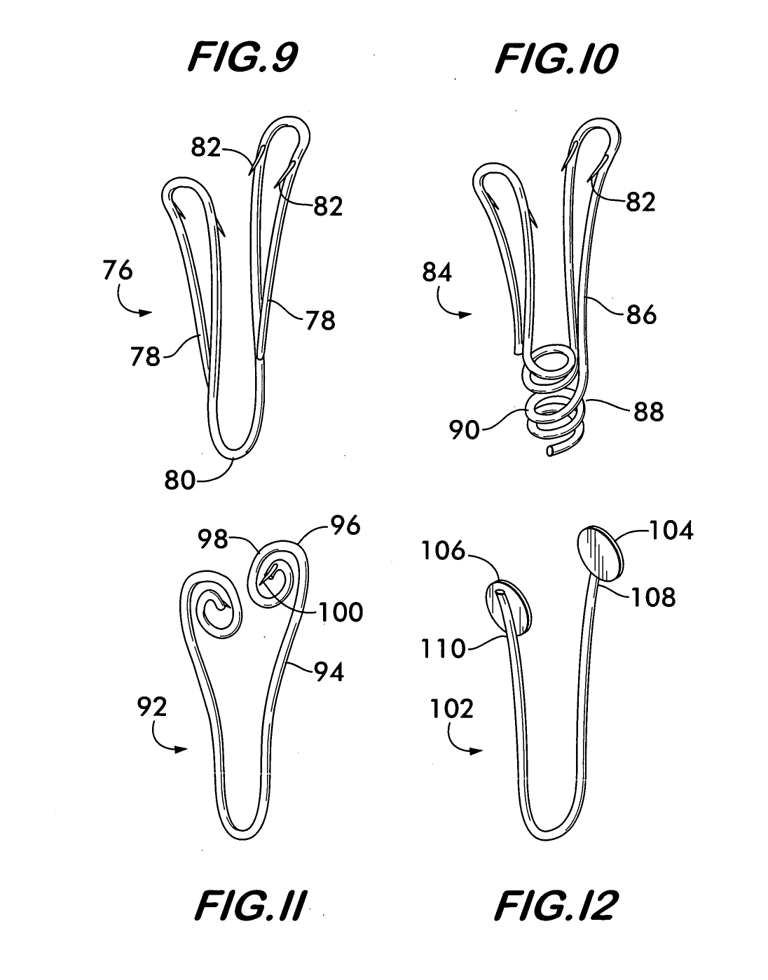

FIG. 9 illustrates a clip 76, wherein legs 78 are formed of elongated wire loops with a simple reverse bend of the wire comprising common attachment 80. The wire looped legs 78 provide multiple attachment points to the valve leaflets through the use of hooks 82 positioned on the various runs of the looped legs 78. As with the other multiple leg embodiments, multiple attachment points reduce the load seen by any one attachment and provide for a more reliable attachment of the clip to the valve. In a variation of the looped leg embodiment, FIG. 10 shows a clip 84 having looped legs 86 and a common attachment 88 formed by helically coiling the wire in a plurality of loops 90. Helically coiled common attachment 88 is adapted to receive the threaded tip 30 of the wire insertion device 32 (see FIG. 2B) to temporarily mount the clip 84 on the insertion device for installing the clip on a valve.

FIG. 11 shows a wire clip 92 having legs 94 with free ends 96 formed of spiral shaped segments 98 with hooks 100 positioned at the end of the wires forming the spiral segments 98. Spiral segments 98 provide increased flexibility at the point of engagement of the hooks 100 and the valve tissue allowing for stress relief of the legs at the attachment points.

FIG. 12 shows a clip 102 having magnets 104 and 106 positioned on the free ends 108, 110 of the clip. The magnets are arranged in a complementary manner so that the north pole of magnet 104 will engage the south pole of magnet 106 and vice versa so that the magnets attract one another to clamp the clip 102 in place on the leaflets of the mitral valve.

FIG. 13 discloses a clip 112 having a common attachment comprising a tubular ferrule 114 from which legs 116 and 118 extend. The legs are formed of a flexible mesh that defines a plurality of interstices 117. Legs 116 and 118 have a fan shape at the free ends 120 and 122, with hooks 124 distributed across the width of the fan shape to provide multiple points of engagement between the legs and the tissue of the mitral valve leaflets. Legs 116 and 118. are also flexibly biased toward one another so that they clamp the leaflets between them when the clip is installed. The size of interstices 117 may be controlled to provide a desired stiffness to the legs 116, 118.

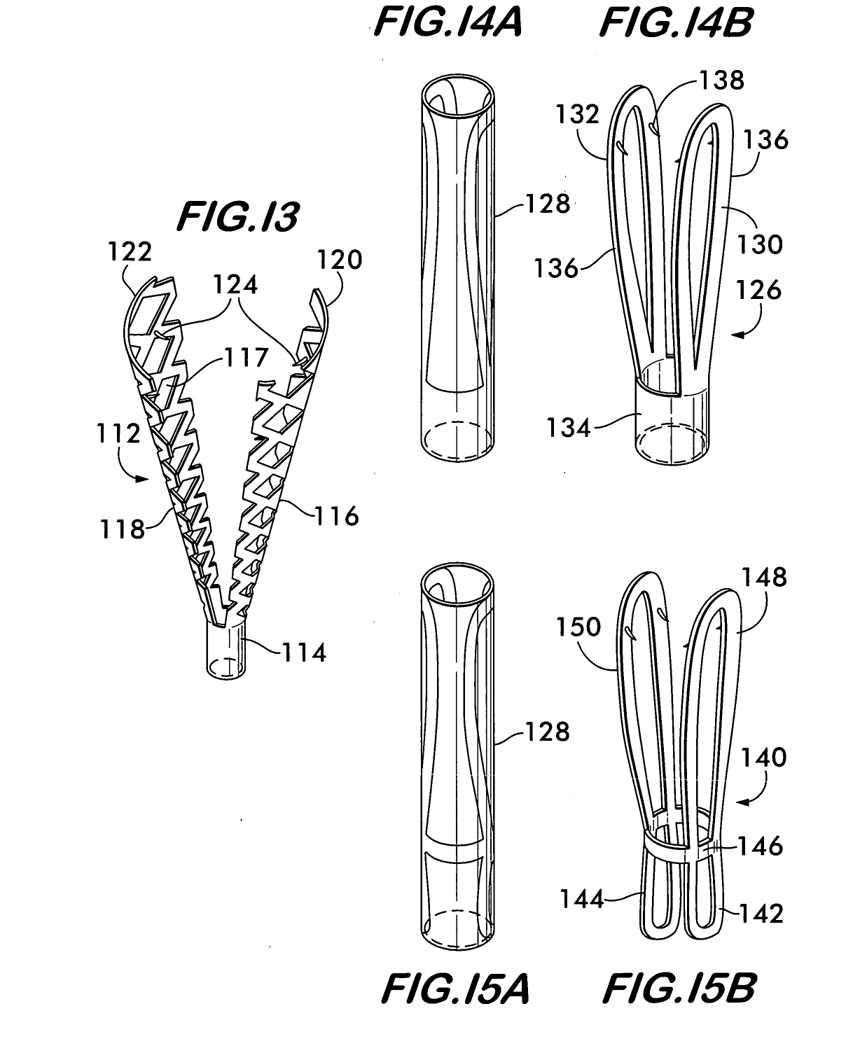

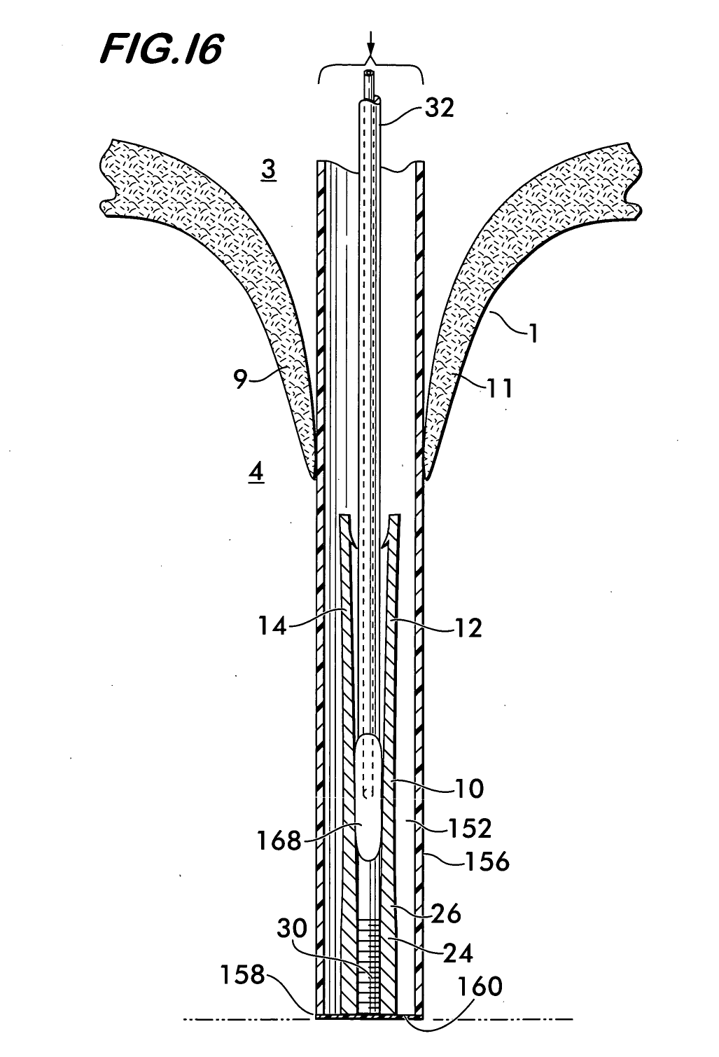

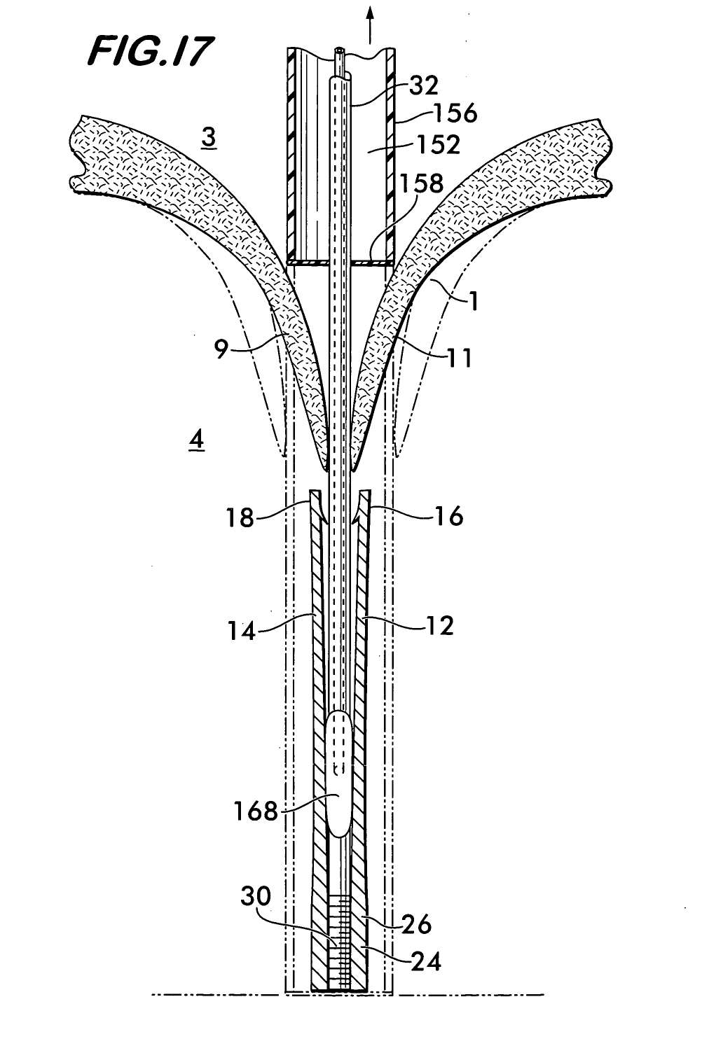

FIGS. 14A and 14B show a clip 126 formed by laser cutting a tube blank 128 to leave legs 130 and 132 attached to a common attachment 134 comprising a cylindrical shell. The legs may be any shape and are shown as loops 136 with multiple hooks 138 positioned on the runs of the loops.

FIGS. 15A and 15B show a clip embodiment 140 formable from a tube blank 128. Clip 140 has a common attachment in the form of a ring 146 from which extend flexible, resilient legs 148 and 150. The legs are positioned lengthwise facing one other, diametrically opposed on ring 146. Actuating projections 142 and 144 extend from the ring 146 in a direction opposite to legs 148 and 150. The actuating projections are substantially aligned with the legs so that, by moving the actuating projections inwardly toward or outwardly apart from one another, the attachment legs 148 and 150 may be respectively separated from one another to receive the mitral valve leaflets or forcibly brought together to clamp down on the leaflets and set the hooks securely in the tissue. Movement of the actuating projections may be effected by, for example, a balloon or a mechanism.

FIGS. 16-20 illustrate the steps of a method for installing the clip 10 according to the invention, it being understood that any of the clip embodiments disclosed herein are useable with the method disclosed with only minor modifications to the method steps necessary to take into account the variations in clip design.

As shown in FIG. 16, the ferrule 26 of clip 10 is screwed onto the threaded tip 30 of the wire insertion device 32 and the assembly is positioned within the lumen 152 of a catheter 156. Catheter 156 is then inserted into the heart percutaneously through the vascular system of the patient. At its tip 158, the catheter 156 has a radiopaque marker 160 permitting fluoroscopic visualization of the position of the catheter tip. The tip 158 is moved through the left atrium 3, past the mitral valve 1 and into the left ventricle 4 as shown in FIG. 16.

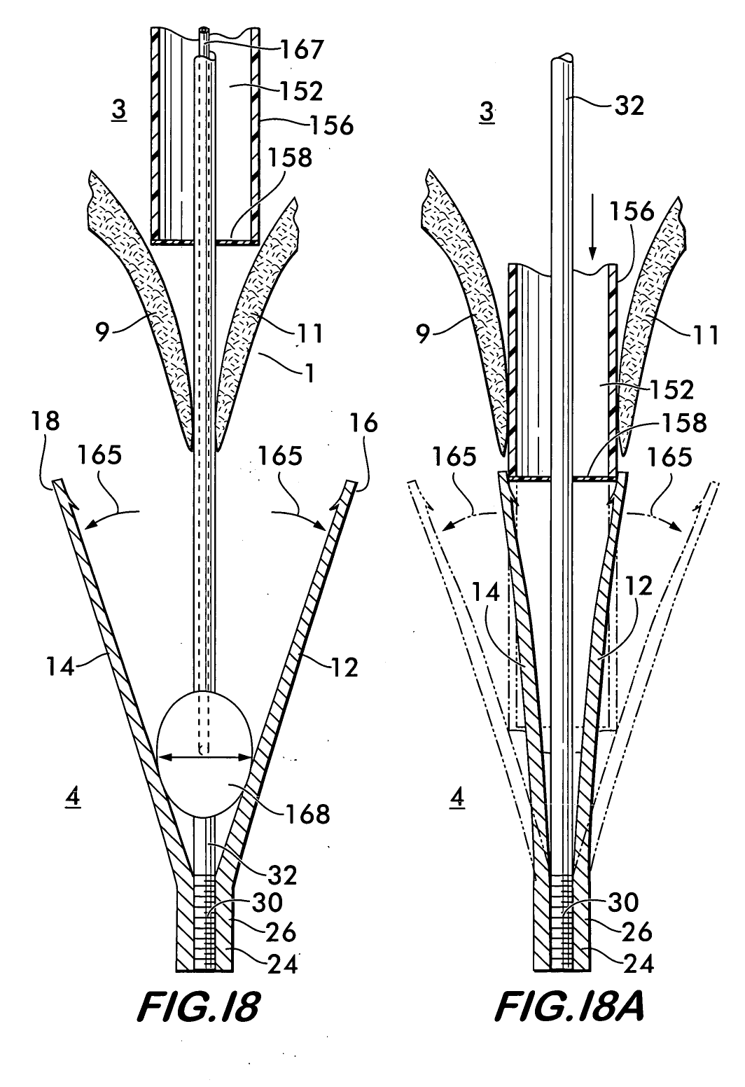

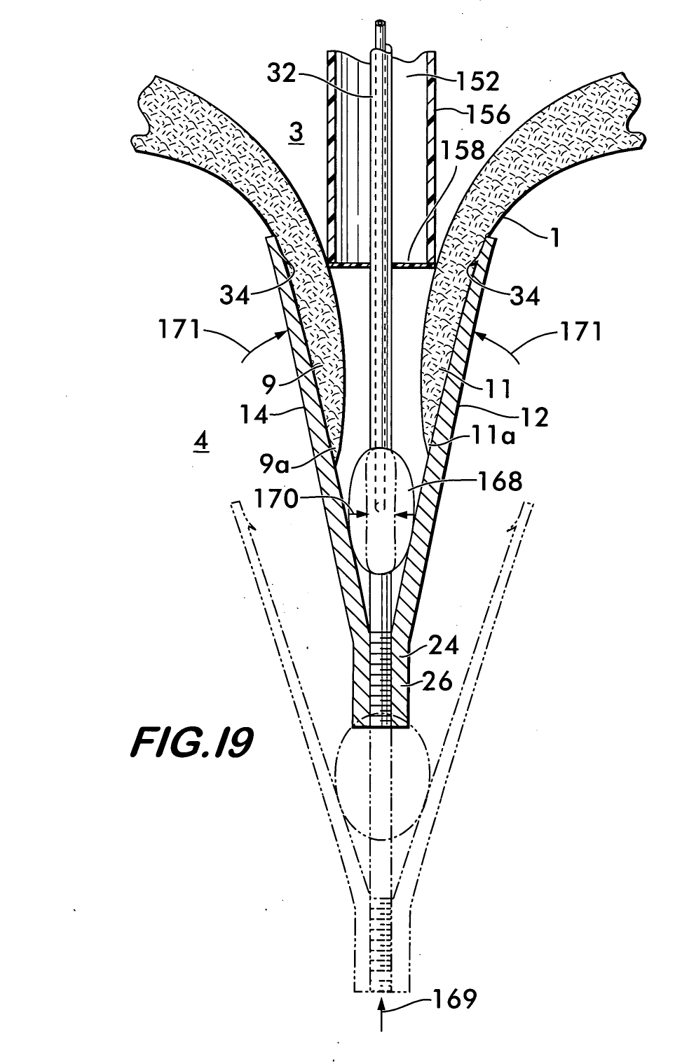

In the next step, shown in FIG. 17, the catheter 156 is withdrawn from the left ventricle 4 into the left atrium 3, but the wire insertion device 32 is simultaneously extended so as to remain within the left ventricle, positioning the clip 10 adjacent to the mitral valve 1. As shown in FIG. 18, the legs 12 and 14 of clip 10, normally biased toward each other, are separated as shown by arrows 165 to position the free ends 16 and 18 straddling the mitral valve 1. The preferred means for separating the legs is by a non-compliant balloon 168 which is positioned on the insertion device 32 adjacent to the threaded tip 30 and inflated hydraulically via a conduit 167 positioned coaxially within the insertion device 32. FIG. 18A shows an alternate means for separating the legs 12 and 14 by forcing the tip 158 of the catheter between the legs. The alternate separation means requires that the legs have some degree of biasing away from one another so that when the clip is extended out of the lumen 152 the legs separate sufficiently so that the catheter tip 158 can be positioned between the legs 12 and 14 by relative motion between the insertion device 32 and the catheter 156.

As shown in FIG. 19, once the legs 12 and 14 are separated (shown in broken line), the insertion device 32 is drawn toward the mitral valve 1 as indicated by arrow 169 so that the leaflets 9 and 11 are between the legs 12 and 14, each leg being adjacent to a respective leaflet. Preferably, the common attachment 24 (threaded ferrule 26) is positioned adjacent to the free edges 9a and 11a of the mitral valve leaflets 9 and 11. Balloon 168 is then deflated as indicated by arrows 170, and the legs, being resiliently biased, move toward one another as indicated by arrows 171 in conformance with their biasing. Leg 12 engages leaflet 11 and leg 14 engages the opposing leaflet 9 and the hooks 34 on each leg dig into the leaflet tissue and fix the clip 10 to mitral valve 1.

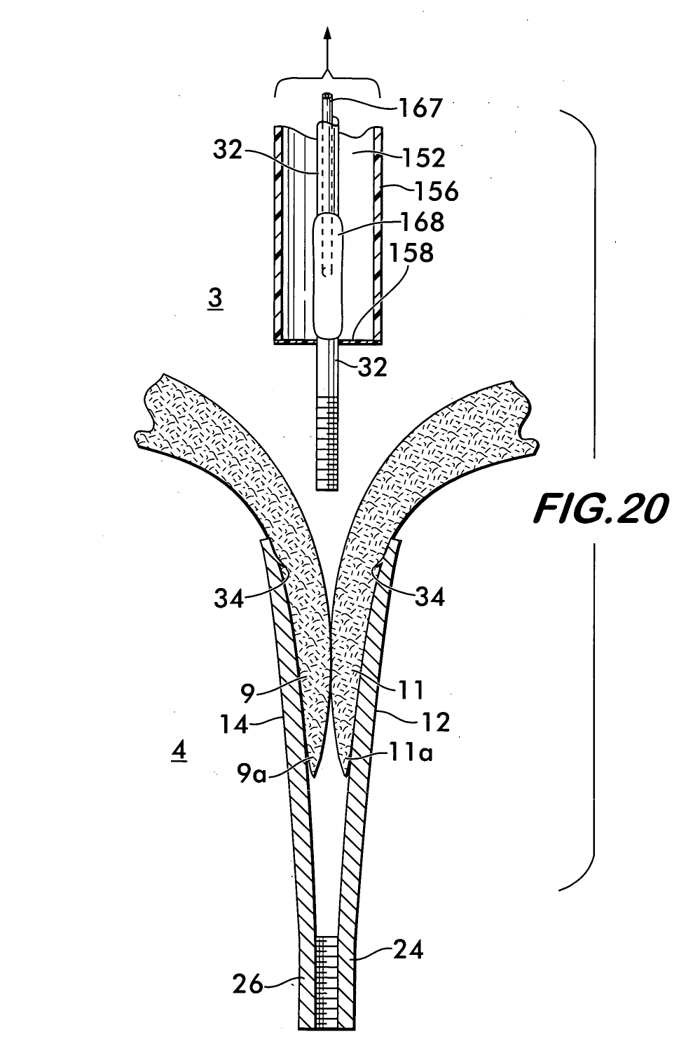

The insertion device 32 is rotated about its long axis, unscrewing the threaded tip 30 from the threaded ferrule 26 and, thus, separating the insertion device 32 from the clip 10. The catheter 156 and the insertion device 32 may then be withdrawn as shown in FIG. 20. The clip is positioned approximately in the center of the mitral valve 1 and effects a repair of the valve in the manner of an Alfieri leaflet stitch by restraining the relative motion of the leaflets 9a and 11a substantially at a point, preventing valve prolapse and blood regurgitation from the left ventricle 4 into the left atrium 3.

Use of the clip 10 or any of its various embodiments offers an improved device and method for repair of mitral valves since the clips as disclosed herein may be used with minimally invasive endovascular procedures which do not require that the patient be put on cardiopulmonary by pass and avoid the trauma of open heart surgery.

What is claimed is:

1. A clip for repair of a heart valve having leaflets, said clip substantially restraining the motion of the leaflets, said clip comprising:
a plurality of resilient, flexible legs arranged substantially lengthwise adjacent to one another, each of said legs having a first end adapted to attach to one of the leaflets and a second end opposite said first end; and
a common attachment located at said second ends of said legs, said second ends being attached to said common attachment, said first ends of said legs being flexibly movable away from one another and capable of receiving the leaflets therebetween, said legs further being resiliently biased toward each other for engagement of each of said first ends with one of the leaflets for attaching said clip thereto.

2. A clip according to claim 1, wherein said common attachment comprises an elongated ferrule.

3. A clip according to claim 2, wherein said elongated ferrule comprises a bore extending lengthwise therethrough.

4. A clip according to claim 3, wherein said bore is threaded.

5. A clip according to claim 1, wherein said common attachment comprises a wire segment formed in a reverse bend.

6. A clip according to claim 5, wherein said legs comprise additional wire segments integrally formed with and extending from said wire segment forming said reverse bend.

7. A clip according to claim 1, wherein said common attachment comprises a wire segment formed into at least one loop.

8. A clip according to claim 7, wherein said loop is oriented substantially parallel to a plane defined by said legs.

9. A clip according to claim 7, wherein said legs comprise additional wire segments integrally formed with and extending from said wire segment forming said loop.

10. A clip according to claim 1, further comprising a plurality of hooks, at least one of said hooks being attached to said first ends of each of said legs and projecting outwardly therefrom, said hooks for engaging said leaflets for attaching said legs thereto.

11. A clip according to claim 1, further comprising a substrate attached to at least one of said legs, said substrate being engageable with said leaflets and having pores therein to facilitate tissue growth from said leaflets onto said substrate for attachment of said legs to said leaflets.

12. A clip according to claim 1, wherein each of said legs comprises an elongated wire loop.

13. A clip according to claim 1, wherein at least one of said legs comprises a spiral-shaped wire segment positioned at its free end, a hook being positioned on said segment and extending outwardly therefrom, said hook being engageable with said leaflet for attaching said leg thereto.

14. A clip according to claim 1, further comprising a pair of magnets, each one of said magnets being positioned substantially at said first end of one of said legs, said magnets being positionable in opposition to one another on opposite sides of said leaflets, said magnets being attracted to one another for securing said clip thereto.

15. A clip according to claim 1, wherein said common attachment comprises a cylindrical shell.

16. A clip according to claim 15, wherein said legs comprise a flexible mesh defining a plurality of interstices positioned along said legs.

17. A clip for repair of a heart valve having leaflets, said clip substantially restraining the motion of the leaflets, said clip comprising:
a ring;
a pair of resilient, flexible legs arranged substantially lengthwise facing one another, each of said legs having a first end adapted to attach to one of the leaflets and a second end attached to said ring, said legs being attached to said ring substantially diametrically opposite to one another;
a pair of actuating projections attached to said ring substantially diametrically opposite to one another, each of said actuating projections being substantially aligned with one of said legs and extending in a direction opposite thereto, said first ends of said legs being flexibly movable toward and away from one another for receiving the leaflets therebetween when said actuating projections are moved respectively away and toward each other.

18. A clip according to claim 17, wherein each of said legs comprise an elongated closed loop extending from said ring.

19. A clip according to claim 17, wherein each of said legs has a hook mounted substantially adjacent to said first end, said hooks adapting said legs for attachment to said leaflets.

20. A method of repairing a heart valve having leaflets by restraining the motion of the leaflets substantially at a point, said method comprising the steps of:
providing a clip comprising a plurality of resilient, flexible legs arranged substantially lengthwise adjacent to one another and resiliently biased toward one another, each of said legs having a first end adapted to attach to one of the leaflets and a second end opposite said first end, said clip further comprising a common attachment located at said second ends of said legs, said second ends being attached to said common attachment;
flexibly moving said first ends of said legs away from one another;
positioning said legs with the leaflets therebetween and said common attachment adjacent to said leaflets; and
allowing each of said first ends to be biased into engagement with one of the leaflets and thereby attaching said clip to the valve, said common attachment being positioned substantially adjacent to the leaflets and defining the point at which the motion of the leaflets is substantially restrained.
